(19) United States
(12) Patent Application Publication
(10) Pub. No.: US 2010/0168165 A1
(43) Pub. Date: Jul. 1, 2010

(54) METHOD OF TREATING DISEASE INVOLVING MYELIN AND/OR AXONAL LOSS

(75) Inventors: Jacqueline A. Quandt, Vancouver (CA); James B. Mitchell, Damascus, MD (US); Anastasia Sowers, Purcellville, VA (US); Murali Krishna Cherukuri, Gaithersburg, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The United States of America, as represented by the Secretary, Dept of Health and Human Services, Bethesda, MD (US)

(21) Appl. No.: 12/706,068

(22) Filed: Feb. 16, 2010

Related U.S. Application Data
(63) Continuation-in-part of application No. PCT/US2008/073007, filed on Aug. 13, 2008.
(60) Provisional application No. 60/955,731, filed on Aug. 14, 2007.

(51) Int. Cl. A61K 31/45; A61P 25/00; A61P 29/00; A61P 37/00

(52) U.S. Cl. 514/315

(57) ABSTRACT

Disclosed is a method of treating a disease involving myelin and/or axonal loss, such as a demyelinating disease, in a mammal comprising administering a compound of formula I

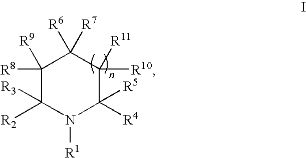

in which R¹-R¹¹ and n are defined herein. Also disclosed are methods of using a compound of formula I to treat neurodegeneration associated with inflammation and to reduce myelin and/or axonal loss.

### CROSS-REFERENCE TO RELATED APPLICATIONS

This patent application is a continuation-in-part of PCT/US2008/073007, filed Aug. 13, 2008, which claims the benefit of U.S. Provisional Patent Application No. 60/955,731, filed Aug. 14, 2007, both of which are incorporated by reference.

### BACKGROUND OF THE INVENTION

Several demyelinating diseases are known. For example, multiple sclerosis (MS) is considered to be an autoimmune demyelinating disease of the central nervous system (CNS) affecting approximately 400,000 Americans with varying degrees of disability. MS can cause problems with muscle control and strength, vision, balance, sensation, and mental functions, and ultimately leaves many individuals wheelchair bound. Therapies have been proposed to treat demyelinating diseases. Some of the proposed therapies attempt to modulate the autoimmune and inflammatory components of the disease, but they are only moderately successful and afford little protection once subsequent damage to the nervous system has occurred. Accordingly, there is a desire for alternative therapies that modulate the autoimmune and inflammatory components and also provide protection against subsequent damage to the nervous system.

### BRIEF SUMMARY OF THE INVENTION

The invention provides a method of treating a disease involving myelin and/or axonal loss, specifically a demyelinating disease, in a mammal comprising administering a compound of formula I

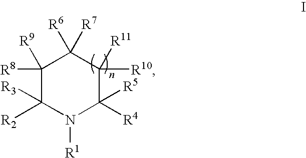

in which R¹-R¹¹ and n are defined herein. The disease can be, for example, multiple sclerosis (MS), optic neuritis, Devic's disease (neuromyelitis optica), transverse myelitis, acute MS (Marburg variant), Balo's concentric sclerosis, Guillain-Barré syndrome, acute disseminated encephalomyelitis (ADEM), adrenoleukodystrophy, or adrenomyeloneuropathy.

The invention further provides methods of using a compound of formula I to treat neurodegeneration associated with inflammation and to reduce myelin and/or axonal loss.

### BRIEF DESCRIPTION OF THE SEVERAL VIEWS OF THE DRAWING(S)

FIG. 1 is a graph illustrating the assessed disease severity of actively induced chronic progressive experimental autoimmune encephalomyelitis (EAE) versus number of days post immunization in control—() and Tempol—(O) fed mice in an embodiment of the invention.

FIG. 2 is a graph illustrating the assessed disease severity of passively induced chronic progressive EAE versus number of days post T cell transfer in control—() and Tempol—(O) fed mice in an embodiment of the invention.

FIG. 3 is a graph illustrating the EAE score versus number of days post T cell transfer in control-fed (♦) mice and mice fed Tempol 2 weeks prior to T cell transfer (), at T cell transfer (▴), and at disease onset (□) in an embodiment of the invention.

FIG. 4 is a graph illustrating the EAE score versus number of days post T cell transfer in control—(♦) and Tempol—(▪) fed mice in an embodiment of the invention. Animals are fed either Tempol or control feed once 60% of the animals exhibit clinical signs of at least a grade 1 (limp tail or greater) post-EAE induction.

FIG. 5 is a bar graph illustrating the relative amount of spinal cord neurofilament H in control—(hatch marked bar) and Tempol—(solid bar) fed mice in an embodiment of the invention.

FIG. 6 is a graph illustrating the cumulative disease score versus the relative amount of neurofilament H in control—(♦) and Tempol—(□) fed mice in an embodiment of the invention.

FIG. 7 is a bar graph illustrating the average T2 relaxation times of control animals (black bar), control-fed EAE animals (light gray bar), and Tempol-fed EAE animals (dark gray bar) in an embodiment of the invention.

FIG. 8 is a bar graph illustrating the average degree of leukocyte infiltration in control—(hatch marked bar) and Tempol—(solid bar) fed mice in an embodiment of the invention.

FIG. 9 is a bar graph illustrating T cell proliferation in control—(gray bar) and Tempol—(black bar) fed mice in an embodiment of the invention.

### DETAILED DESCRIPTION OF THE INVENTION

Nitroxide compounds that comprise or yield a stable radical, such as Tempol, are anti-oxidants that can scavenge free radicals that can mediate tissue damage and destruction. It is contemplated that a nitroxide compound of formula I can serve therapeutically at both the autoimmune/inflammatory and neurodegenerative stages of a disease involving impaired myelin and/or axons, e.g., a demyelinating disease, thereby acting to limit (i) the generation of autoimmune responses (e.g., reduce an immune response) and/or (ii) damage to the nervous system itself.

Accordingly, the present invention provides a method of treating a disease involving myelin and/or axonal loss in a mammal comprising administering to the mammal an effective amount of a compound of formula I

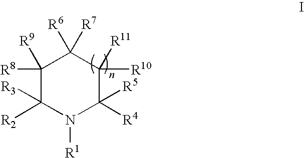

wherein

R¹ is selected from the group consisting of OH, OZ, O., and ═O,
wherein Z is selected from the group consisting of C₁₋₁₂ alkyl, C₂₋₁₂ alkenyl, C₃₋₈ cycloalkyl, C₃₋₈ heterocycloalkyl, and C₆₋₃₀ aryl;

R², R³, R⁴, and R⁵ are the same or different and are selected from the group consisting of hydrogen, C₁₋₁₂ alkyl, C₂₋₁₂ alkenyl, and C₂₋₁₂ alkynyl;

R⁶ and R⁷ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino, or

R⁶ and R⁷ together form ═O;

R⁸, R⁹, R¹⁰, and R¹¹ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino;

optionally one of R⁶ and R⁷ and one of R⁸ and R⁹ can be absent such that a double bond joins the two carbon atoms to which the remaining of one of R⁶ and R⁷ and one of R⁸ and R⁹ are attached; and

n is 0 or 1;

provided that the disease is not ataxia telangiectasia (AT).

In a specific embodiment, the disease is a demyelinating disease and/or involves axonal loss, e.g., damage and/or impairment (via demyelination or other pathways). The disease can involve a loss or defect of oligodendrocyte. The disease can be an inflammatory disease (e.g., an inflammatory demyelinating disease and/or an inflammatory disease with myelin or axonal loss (e.g., damage and/or impairment)). For example, the method of treatment can include the aspect in which the autoimmune component of the inflammatory demyelinating disease is treated (e.g., immune activation is reduced).

The disease is any disorder that involves myelin and/or axonal loss (e.g., damage, and/or impairment), regardless of the cause (e.g., a demyelinating disease). A demyelinating disease is any disorder that results in deficient or abnormal myelination (e.g., destruction of myelin). The pathology of the demyelinating disease can have autoimmune, inflammatory, neurodegenerative, or other components. In an embodiment, the disease is classified as an inflammatory demyelinating disease or an autoimmune/inflammatory demyelinating disease. The disease can be, for example, multiple sclerosis (MS), optic neuritis, Devic's disease (neuromyelitis optica), transverse myelitis, acute MS (Marburg variant), Balo's concentric sclerosis, Guillain-Barré syndrome, acute disseminated encephalomyelitis (ADEM), adrenoleukodystrophy, or adrenomyeloneuropathy. In a preferred embodiment, the disease is multiple sclerosis (MS). The hallmark characteristics of MS include the breakdown of the blood brain barrier, leukocytic infiltration, and demyelination often associated with axonal loss (e.g., damage and/or impairment).

In accordance with the invention, treatment of the disease (e.g., a demyelinating disease) includes reduction (including complete or partial reduction) in the incidence and/or severity of the disease, including a reduction in a variety of symptoms, e.g., paresis, paralysis, spasticity, ataxia (e.g., cerebellar ataxia, sensory ataxia, and/or vestibular ataxia, including, spinocerebellar ataxia, episodic ataxia, dentatorubropallidoluysian ataxia, Friedreich's ataxia, Niemann Pick disease, and abetalipoproteinaemia) and/or tremor.

The invention further provides a method of treating neurodegeneration associated with (e.g., induced by) inflammation, in a mammal comprising administering to the mammal an effective amount of a compound of formula I

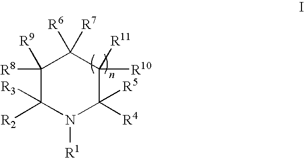

wherein

R¹ is selected from the group consisting of OH, OZ, O, and ═O,
wherein Z is selected from the group consisting of C₁₋₁₂ alkyl, C₂₋₁₂ alkenyl, C₃₋₈ cycloalkyl, C₃₋₈ heterocycloalkyl, and C₆₋₃₀ aryl;

R², R³, R⁴, and R⁵ are the same or different and are selected from the group consisting of hydrogen, C₁₋₁₂ alkyl, C₂₋₁₂ alkenyl, and C₂₋₁₂ alkynyl;

R⁶ and R⁷ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino, or

R⁶ and R⁷ together form ═O;

R⁸, R⁹, R¹⁰, and R¹¹ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino;

optionally one of R⁶ and R⁷ and one of R⁸ and R⁹ can be absent such that a double bond joins the two carbon atoms to which the remaining of one of R⁶ and R⁷ and one of R⁸ and R⁹ are attached; and

n is 0 or 1;

provided that the neurodegeneration is not caused by ataxia telangiectasia (AT).

The term “associated with” includes instances in which neurodegeneration and inflammation are both detected. The inflammation may or may not induce the neurodegeneration. In a preferred embodiment, the inflammation induces the neurodegeneration. The method includes treating neurodegeneration induced by an immune response that is also associated with demyelination. In an embodiment of this method, the compound of formula I provides protection against onset and progression of neurodegeneration of the central nervous system (CNS). The treatment of the neurodegradation associated with inflammation and/or an immune response that is also associated with demyelination includes reduction (including complete or partial reduction) in the incidence and/or severity of the disease, including a reduction in a variety of symptoms, e.g., paresis, paralysis, spasticity, ataxia, and/or tremor.

The treatment of neurodegeneration includes neurodegeneration that is caused by a disease such as multiple sclerosis (MS), optic neuritis, Devic's disease (neuromyelitis optica), transverse myelitis, acute MS (Marburg variant), Balo's concentric sclerosis, Guillain-Barré syndrome, acute disseminated encephalomyelitis (ADEM), adrenoleukodystrophy, or adrenomyeloneuropathy. In an embodiment, the neurodegeneration is caused by multiple sclerosis (MS).

Experimental Autoimmune Encephalomyelitis (EAE) is an acute or chronic-relapsing, acquired, inflammatory, and demyelinating autoimmune disease model (see, e.g., Wekerle et al., Ann. Neurol., 36: S47-S53 (1994); Goverman et al., Lab Anim. Sci., 46: 482-92 (1996); Bischof et al., Proc. Natl. Acad. Sci. USA, 98: 12168 (2001)). In the model, the animal is injected with whole (or parts of) various proteins that make up myelin, the insulating sheath that surrounds nerve cells (neurons). These proteins induce an autoimmune response in the animal, such that the animal's immune system mounts an attack on its own myelin as a result of exposure to the injection. As a result, the animal develops a disease process that closely resembles MS, particularly in humans. It has been surprisingly found that Tempol, a compound of formula I, in an embodiment does not specifically prevent the generation of autoimmune T cells and yet limits the damage that autoimmune T cells primed in the same or other animals can cause when they access the nervous system.

Any suitable mammal can be used in the EAE model, such as mice, rats, guinea pigs, rabbits, macaques, rhesus monkeys, and marmosets. Rodents, such as mice and rats are particularly preferred given the resemblance of the induced disease to MS in humans.

Several proteins or parts of proteins (antigens) can be used to induce EAE, including myelin basic protein (MBP), proteolipid protein (PLP), and myelin oligodendrocyte glycoprotein (MOG). MBP, PLP, and MOG can be synthesized by using, for example, standard 9-fluorenylmethoxycarbonyl (Fmoc) chemistry on a protein synthesizer and then purified by conventional techniques (e.g., high performance liquid chromatography (HPLC)). Literature methods are known (see, e.g., Fridkis-Hareli et al., J. Clin. Invest., 109: 1635-1643 (2002)). Alternatively, these proteins are commercially available (e.g., AnaSpec, San Jose, Calif.; GenScript, Piscataway, N.J.).

It has been found that a compound of formula I reduces the degree of edema associated with inflammation and tissue damage in passively-induced EAE. A compound of formula I also limits the degree of leukocytic infiltration in actively-induced EAE, thereby reducing inflammation. Thus, the invention contemplates reducing edema and/or inflammation in a mammal comprising administration of a compound of formula I, as described herein. These results also indicate that oligodendrocytes are protected from edema and inflammation with a compound of formula I. The protection of the oligodendrocytes that produce myelin that coat the axon translates into the ability to reduce myelin and/or axonal loss. Accordingly, in an embodiment, the present invention provides a method of reducing myelin and/or axonal loss in a mammal comprising administering to the mammal an effective amount of a compound of formula I

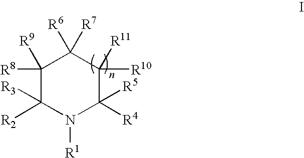

wherein

R¹ is selected from the group consisting of OH, OZ, O, and ═O,
wherein Z is selected from the group consisting of C₁₋₁₂ alkyl, C₂₋₁₂ alkenyl, C₃₋₈ cycloalkyl, C₃₋₈ heterocycloalkyl, and C₆₋₃₀ aryl;

R², R³, R⁴, and R⁵ are the same or different and are selected from the group consisting of hydrogen, C₁₋₁₂ alkyl, C₂₋₁₂ alkenyl, and C₂₋₁₂ alkynyl;

R⁶ and R⁷ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino, or

R⁶ and R⁷ together form ═O;

R⁸, R⁹, R¹⁰, and R¹¹ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino;

optionally one of R⁶ and R⁷ and one of R⁸ and R⁹ can be absent such that a double bond joins the two carbon atoms to which the remaining of one of R⁶ and R⁷ and one of R⁸ and R⁹ are attached; and

n is 0 or 1.

In accordance with an embodiment of the invention, the compounds of formula I provide protection against disorders associated with myelin and/or axonal loss, myelin and/or axonal loss per se, and/or neurodegeneration associated with inflammation without abolishing the generation of myelin-specific T cells.

In any of the inventive methods described herein, R¹ preferably is O. (a radical). R², R³, R⁴, and R⁵ preferably are C₁₋₁₂ alkyl, particularly a C₁₋₄ alkyl (e.g., methyl, ethyl, n-propyl, i-propyl, n-butyl, sec-butyl, i-butyl, t-butyl). In some embodiments, R⁶ and R⁷ are the same or different and are selected from the group consisting of hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, alkylsulfonyloxy, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino. R⁸, R⁹, R¹⁰, and R¹¹ preferably are the same or different and each is hydrogen, halogen, hydroxyl, or C₁₋₁₂ alkyl. In a particular embodiment, R⁷, R⁸, R⁹, R¹⁰, and R¹¹ are hydrogen.

R⁷ preferably is hydrogen, halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, alkylsulfonyloxy, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, or haloalkylcarbonylamino. R⁷ preferably is hydrogen, halogen, hydroxyl, or C₁₋₁₂ alkyl, particularly hydrogen, halogen, or C₁₋₁₂ alkyl.

R⁶ preferably is halogen, hydroxyl, thiol, cyano, isothiocyanato (—NCS), C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, alkylsulfonyloxy, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, or haloalkylcarbonylamino. Preferably, R⁶ is hydrogen, hydroxyl, C₁₋₁₂ alkyl, C₁₋₁₂ alkoxy, cyano, isothiocyanato, amino, carboxy, alkylcarbonylamino, haloalkylcarbonylamino, or alkylsulfonyloxy. More preferably R⁶ is hydroxyl, C₁₋₁₂ alkyl, C₁₋₁₂ alkoxy, cyano, isothiocyanato, alkylcarbonylamino, haloalkylcarbonylamino, or alkylsulfonyloxy.

When n is 0, the compound of formula I is a 5-membered ring, and the substituents R¹⁰ and R¹¹ are absent. When n is 1, the compound of formula I is a 6-membered ring. In a preferred embodiment, n is 1.

In an embodiment, the compound of formula I is 4-hydroxy-2,2,6,6-tetramethylpiperidine-1-oxyl (Tempol, also known as 4-hydroxy-Tempo).

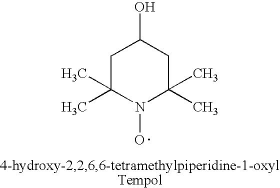

Examples of compounds of formula I also include 2,2,6,6-tetramethylpiperidine-1-oxyl (Tempo), 4-methoxy-2,2,6,6-tetramethyl-1-piperidine-1-oxyl, 4-carboxy-2,2,6,6-tetramethyl-1-piperidine-1-oxyl, 4-oxo-2,2,6,6-tetramethyl-1-piperidine-1-oxyl, 4-amino-2,2,6,6-tetramethyl-1-piperidine-1-oxyl, 4-cyano-2,2,6,6-tetramethyl-1-piperidine-1-oxyl, 4-isocyanato-2,2,6,6-tetramethyl-1-piperidine-1-oxyl, 4-acetamido-2,2,6,6-tetramethyl-1-piperidine-1-oxyl, 4-(2-bromoacetamido)-2,2,6,6-tetramethyl-1-piperidine-1-oxyl, 4-(2-chloroacetamido)-2,2,6,6-tetramethyl-1-piperidine-1-oxyl, 4-(2-iodoacetamido)-2,2,6,6-tetramethyl-1-piperidine-1-oxyl, and 4-(methylsulfonyloxy)-2,2,6,6-tetramethyl-1-piperidine-1-oxyl, particularly 4-methoxy-2,2,6,6-tetramethyl-1-piperidine-1-oxyl, 4-oxo-2,2,6,6-tetramethyl-1-piperidine-1-oxyl, 4-cyano-2,2,6,6-tetramethyl-1-piperidine-1-oxyl, 4-isocyanato-2,2,6,6-tetramethyl-1-piperidine-1-oxyl, 4-acetamido-2,2,6,6-tetramethyl-1-piperidine-1-oxyl, 4-(2-bromoacetamido)-2,2,6,6-tetramethyl-1-piperidine-1-oxyl, 4-(2-chloroacetamido)-2,2,6,6-tetramethyl-1-piperidine-1-oxyl, 4-(2-iodoacetamido)-2,2,6,6-tetramethyl-1-piperidine-1-oxyl, and 4-(methylsulfonyloxy)-2,2,6,6-tetramethyl-1-piperidine-1-oxyl.

Referring now to terminology used generically herein, the term “alkyl” implies a straight or branched alkyl moiety containing from, for example, 1 to 12 carbon atoms, preferably from 1 to 8 carbon atoms, more preferably from 1 to 6 carbon atoms. Examples of such moieties include methyl, ethyl, propyl, isopropyl, n-butyl, sec-butyl, isobutyl, text-butyl, pentyl, isoamyl, hexyl, octyl, dodecanyl, and the like.

The term “alkenyl” as used herein refers to a straight chain or branched non-cyclic hydrocarbon moiety having an indicated number of carbon atoms (e.g., C₂-C₂₀, C₂-C₁₀, C₂-C₄, etc.). Examples of such moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, and the like. The double bond of an alkenyl group can be unconjugated or conjugated to another unsaturated group.

The term “alkynyl” as used herein refers to a straight chain or branched non-cyclic hydrocarbon moiety having an indicated number of carbon atoms (e.g., C₂-C₂₀, C₂-C₁₀, C₂-C₆, etc.), and including at least one carbon-carbon triple bond. Examples of such moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. The triple bond of an alkynyl group can be unconjugated or conjugated to another unsaturated group.

The term “cycloalkyl,” as used herein, means a cyclic alkyl moiety containing from, for example, 1-3 rings (i.e., monocyclic, bicyclic, tricyclic, or spiro), 3 to 8 carbon atoms per ring, preferably from 5 to 8 carbon atoms, more preferably from 5 to 6 carbon atoms. Examples of such moieties include cyclopropyl, cyclobutyl, cyclopentyl, cyclohexyl, and the like.

The term “heterocycloalkyl” means a cycloalkyl moiety having one or more heteroatoms, e.g., nitrogen, sulfur, and/or oxygen. Preferably, a heterocycloalkyl is a 5 or 6-membered monocyclic ring and contains one, two, or three heteroatoms selected from the group consisting of nitrogen, oxygen, and/or sulfur. The heterocycloalkyl can be attached to the parent structure through a carbon atom or a heteroatom of the heterocycloalkyl moiety. Examples of such heterocycloalkyl moieties are pyrrolinyl, pyranyl, piperidyl, tetrahydrofuranyl, tetrahydrothiopheneyl, and morpholinyl.

The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, preferably from 6 to 18 carbon atoms, more preferably from 6 to 14 carbon atoms and most preferably from 6 to 10 carbon atoms. It is understood that the term aryl includes carbocyclic moieties that are planar and comprise 4n+2π electrons, according to Hülckel's Rule, wherein n=1, 2, or 3.

The term “halogen” as used herein, means a substituent selected from Group VIIA, such as, for example, fluorine, bromine, chlorine, and iodine.

The term “oxo” as used herein, means the substituent ═O.

The term “alkoxy” embraces an alkyl group attached to an ether oxygen. The alkyl group is the same as described herein. Examples of such a group include methoxy, ethoxy, t-butoxy, and the like.

The term “alkylthio” as used herein, refers to an alkyl group, as described herein, attached to a sulfur moiety. An example of such substituent is ethylthio.

The terms “hydroxyalkyl” and “mercaptoalkyl” refer to an alkyl group that is substituted with —OH or —SH, respectively. The alkyl group is the same as described herein.

The terms “alkylamino” and “arylamino” refer to groups with one hydrogen and one alkyl or aryl group, respectively, directly attached to a trivalent nitrogen atom. The terms “dialkylamino” and “diarylamino” refer to groups with two of the same or different alkyl or aryl groups, respectively, directly attached to a trivalent nitrogen atom.

The term “aminoalkyl” refers to an alkyl group substituted with —NH₂. The terms “alkylaminoalkyl” and “arylaminoalkyl” refer to an alkyl group substituted with —NHR, in which R is an alkyl or aryl group, respectively, as described herein. The term “dialkylaminoalkyl” refers to an alkyl group substituted with —NRR′, in which R and R′ are the same or different alkyl group, as described herein. The term “diarylaminoalkyl” refers to an alkyl group substituted with NRR′, in which R and R′ are the same or different aryl group, as described herein.

The term “carboxy” refers to the group —C(O)OH. The terms “alkylcarbonyl” and “arylcarbonyl” refer to the group —C(O)R, in which R is an alkyl or aryl group, as described herein.

The terms “carboxyalkyl” and “carboxyaryl” refer to an alkyl or aryl group, respectively, substituted with —C(O)OH. The terms “alkylcarbonylalkyl” and “alkylcarbonylaryl” refer to an alkyl or aryl group, respectively, substituted with —C(O)R, in which R is an alkyl, as described herein.

The term “alkylcarbonylamino” refers to the group —NRC(O)R′. The moiety R is a hydrogen or alkyl group and R′ is an alkyl group, as described herein. An example of an alkylcarbonylamino is acetamido.

The term “haloalkylcarbonylamino” refers to the group —NRC(O)R′X, in which R is a hydrogen or alkyl group, R′ is an alkylene, and X is a halogen, as described herein. Examples of such substituent include 2-bromoacetamido, 2-chloroacetamido, 2-iodooacetamido, 2-bromo-N-methyl-acetamido, 3-bromopropanamido, and the like.

The term “alkylsulfonyloxy” refers to the group —OSO₂R, in which R is an alkyl group, as described herein. Examples of such substituent include methylsulfonyloxy and ethylsulfonyloxy.

Whenever a range of the number of atoms in a structure is indicated (e.g., a C₁₋₁₂, C₁₋₈, C₁₋₆, or C₁₋₄ alkyl, alkylamino, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

The compounds of formula I can be prepared by any suitable method. For example, compounds of formula I in which R¹ is O., can be prepared by oxidation of 2,2,6,6-tetramethylpiperidine. See, e.g., Lebelev et al., Zhur. Obshch. Khim., 30: 1631ff (1960). In addition, certain compounds of formula I (e.g., Tempol) are commercially available (e.g., Sigma-Aldrich, St. Louis, Mo.).

Isolation and purification of the compound of formula I can be effected, if desired, by any suitable separation or purification procedure such as, for example, filtration, extraction, crystallization, column chromatography, thin-layer chromatography, thick-layer chromatography, preparative low or high-pressure liquid chromatography, or a combination of these procedures.

The present inventive methods encompass administering a pharmaceutical composition comprising at least one compound of formula I or a pharmaceutically acceptable salt thereof and a pharmaceutically acceptable carrier. The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions; the compounds of the present invention can be formulated as inclusion complexes, such as cyclodextrin inclusion complexes or liposomes.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting. Preferably, the compound of formula I, particularly Tempol, or a composition thereof is administered to the mammal orally.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of formula I, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of formula I can be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

In certain embodiments, the compound of formula I is prepared as an injectable formulation that is free or substantially (e.g., at least 90%, at least 95%, at least 97%, at least 98%, at least 99%) free of a lipid or liposome.

Additionally, the compounds of formula I can be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable carriers and their formulations are further described in A. R. Gennaro, ed., Remington: The Science and Practice of Pharmacy (19th ed.), Mack Publishing Company, Easton, Pa. (1995).

The compound of formula I or a composition thereof can potentially be administered as a pharmaceutically acceptable acid-addition, base neutralized or addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid. The conversion to a salt is accomplished by treatment of the base compound with at least a stoichiometric amount of an appropriate acid. Typically, the free base is dissolved in an inert organic solvent such as diethyl ether, ethyl acetate, chloroform, ethanol, methanol, and the like, and the acid is added in a similar solvent. The mixture is maintained at a suitable temperature (e.g., between 0° C. and 50° C.). The resulting salt precipitates spontaneously or can be brought out of solution with a less polar solvent.

The neutral forms of the compounds can be regenerated by contacting the salt with a base and isolating the parent compound in the conventional manner. The parent form of the compound differs from the various salt forms in certain physical properties, such as solubility in polar solvents, but otherwise the salts are equivalent to the parent form of the compound for the purposes of the present invention.

For purposes of the present invention, the term “mammal” includes, but is not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simioids (monkeys) or of the order Anthropoids (humans and apes). An especially preferred mammal is the human.

The amount or dose of a compound of formula I, a salt thereof, or a composition thereof should be sufficient to affect a therapeutic or prophylactic response in the mammal over a reasonable time frame. The appropriate dose will depend upon the nature and severity of the disease or affliction to be treated or prevented, as well as by other factors. For instance, the dose also will be determined by the existence, nature and extent of any adverse side effects that might accompany the administration of a particular compound or salt. Ultimately, the attending physician will decide the dosage of the compound of formula I with which to treat each individual patient, taking into consideration a variety of factors, such as age, body weight, general health, diet, sex, compound or salt to be administered, route of administration, and the severity of the condition being treated. Typical doses can be, for example, 0.01 mg to 1 g or more daily, such as, e.g., 5 mg to 500 mg daily. Typically, a therapeutic treatment dose can be from about 10 mg or less to about 1 g or more (e.g., about 10 mg to about 500 mg, about 10 mg to about 100 mg). A prophylactic treatment dose can be from about 0.01 mg to about 10 mg (e.g., about 0.1 mg to about 5 mg, about 0.2 mg to about 1 mg). Moreover, the dose can be administered via any suitable manner, as described herein, and including administration with food (e.g., prior to consuming food, concurrently administered with food (including combined with the food itself and/or maintained separately but co-administered), and/or subsequent to consuming food).

The following examples further illustrate the invention but, of course, should not be construed as in any way limiting its scope.

### ABBREVIATIONS

| 4-hydroxy-2,2,6,6-tetramethylpiperidine-N-oxyl | Tempol |
|---|---|
| incomplete Freund's adjuvant | IFA |
| myelin oligodendrocyte glycoprotein | MOG |
| phosphate buffered solution | PBS |
| proteolipid protein | PLP |
| Swiss Jim Lambert | SJL |

### Example 1

This example demonstrates that a nitroxide compound of formula I reduces the incidence and severity of actively- and passively-induced chronic progressive EAE in an embodiment of the invention.

SJL mice are placed on Tempol-containing food two weeks prior to the initiation of experimental autoimmune encephalomyelitis (EAE) either by active immunization with myelin antigens or the transfer of activated encephalitogenic T cells. Animals receive bacon-flavored control food and Tempol-containing food ad libitum (Bio-Serv, Frenchtown, N.J.). The composition of the control food and Tempol-containing food is identical with exception to the addition of Tempol at a concentration of 10 mg/gram of food (Mitchell et al., Free Radic. Biol. Med., 34: 93-102 (2003)). Bacon flavored food is used to mask the taste of Tempol. All experiments with animals are carried out according to protocols approved by and on file with the National Institute of Neurological Disorders and Stoke (NINDS) Animal Care and Use Committee (ACUC).

For the EAE induction, all animals are female, 8-10 weeks of age at time of myelin protein immunization. Animals are immunized for active EAE induction with 200 μg of MOG35-55 peptide in 4 mg/ml H37Ra Mycobacterium/IFA with 200 ng pertussis toxin delivered intraperitoneally (IP) on days 0 and 2. For passive EAE induction, SJL animals are immunized with 75 μg of PLP139-151 peptide in 1 mg/ml H37Ra Mycobacterium/IFA (Difco, Detroit, Mich.). Ten days later, axillary and inguinal lymph nodes are removed and placed into culture with 5 μg/ml of PLP139-151 peptide. After 3 days, activated T cells are counted and 2 million blasted T cells are transferred IP to each SJL recipient mouse.

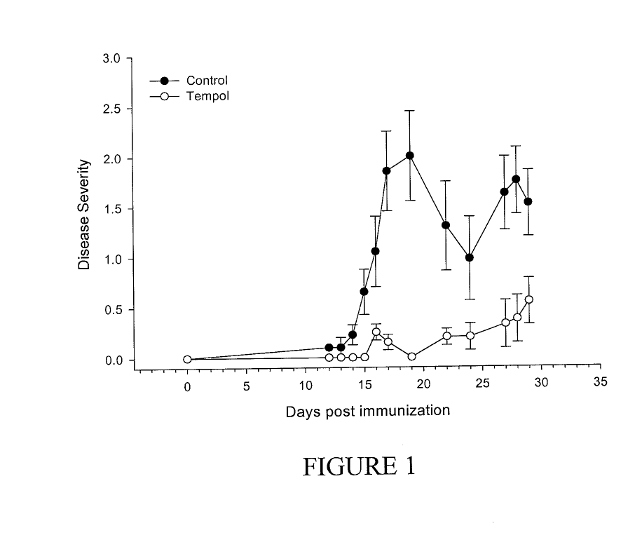

Mice are placed on control- or Tempol-containing food 14 days (T-14) prior to immunization with MOG peptide for EAE induction (day 0=date of immunization). Mice are blindly evaluated for onset of disease and clinical scores are assigned following examination as follows: 0=no clinical signs; 1=flaccid tail; 2=paresis of one or both hind legs; 3=paralysis of one or both hind legs; 4=quadriparesis or quadriparalysis; 5=moribund. The cumulative score represents the mean of the summation of single scores recorded in each mouse over days 0 (day of immunization) to day 30 post disease induction. In this study, mice that are fed Tempol generate myelin antigen-specific T cells which are unable to cause paralytic disease in immunized mice. The results are summarized in Table 1 and FIG. 1. The results indicate that Tempol reduces the incidence and severity of actively induced chronic progressive EAE.

TABLE 1

| | | Average | |
|---|---|---|---|
| | Incidence of | cumulative | Average peak |
| | disease | disease score | disease severity |
| Control-food | .90 | 13.15 | 2.55 |
| n = 10 | | | |
| Tempol-containing food | .30 | 2.05 | 0.83 |
| n = 10 | | | |
| (inventive) | | | |
| | | *p = .001 | *p = .0004 |

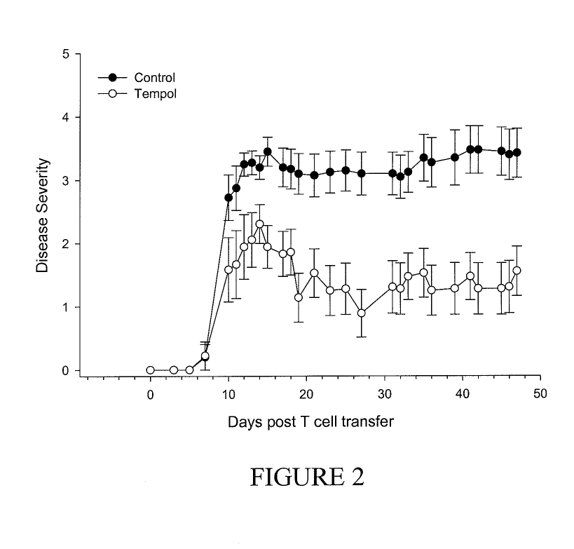

Mice are placed on control- or Tempol-containing food 14 days (T-14) prior to receipt of encephalitogenic T cells (day 0=date of T cell transfer). Mice are blindly evaluated for onset of disease and clinical scores were assigned following examination as follows: 0=no clinical signs; 1=flaccid tail; 2=paresis of one or both hind legs; 3=paralysis of one or both hind legs; 4=quadriparesis or quadriparalysis; 5=moribund. The cumulative score represents the mean of the summation of single scores recorded in each mouse over days 0 (day of T cell transfer) to day 48 post disease induction. PLP-specific T cells are capable of causing severe paralytic disease in the brain and spinal cord of control fed mice, yet Tempol-fed mice are protected showing decreased incidence of disease as well as reduced severity. Control animals on average reached a peak disease of complete hind limb paralysis with forelimb weakness, whereas animals on Tempol feed experienced hind limb paresis at peak disease. The results are summarized in Table 2 and FIG. 2. The results indicate that Tempol reduces the incidence and severity of passively induced chronic progressive EAE, thereby reducing the neurodegenerative effects of PLP-specific T cells.

TABLE 2

| | | Average | |
|---|---|---|---|
| | Incidence of | cumulative | Average peak |
| | disease | disease score | disease severity |
| Control-food | 1.00 | 78.8 | 3.85 |
| n = 10 | | | |
| Tempol-containing food | .70 | 33.9 | 2.81 |
| n = 10 | | | |
| (inventive) | | | |
| | | *p = .00046 | *p = .011 |

### Example 2

This example demonstrates that a nitroxide compound of formula I reduces the severity of and ameliorates disease when administered after the onset of clinical symptoms under both a relapsing remitting model and chronic model of CNS autoimmunity, in accordance with an embodiment of the invention.

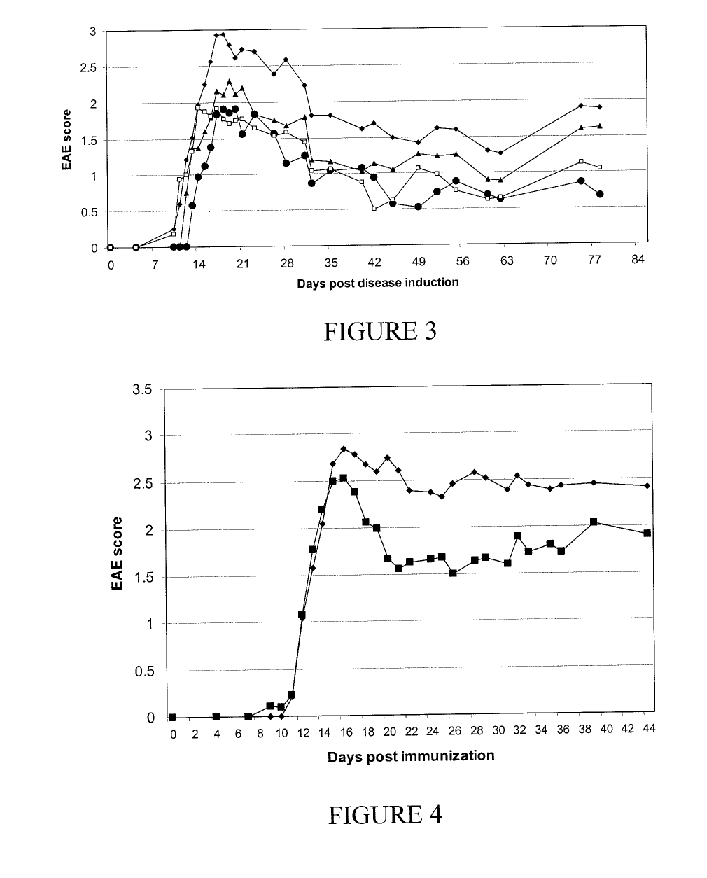

SJL animals are induced for disease by the passive transfer of encephalitogenic T cells as described in Example 1. Animals are placed on either Tempol or control feed at either (i) 2 weeks prior to transfer, (ii) at the time of transfer, or (iii) at the onset of disease. As shown in FIG. 3, Tempol reduces the severity of clinical symptoms and the cumulative disease score regardless of the time of administration. These results suggest that therapeutic treatment is as effective as prophylactic treatment in passive disease induction.

Animals are immunized for MOG-induced active EAE and switched to Tempol or control feed once 60% of the animals exhibit clinical signs of at least a grade 1 (limp tail or greater). As shown in FIG. 4, within 2 days of feed administration, the severity of disease within the Tempol-fed group drops off and is maintained as the animals are maintained on Tempol feed.

### Example 3

This example demonstrates that a nitroxide compound of formula I reduces the degree of neuronal damage and/or loss as indicated by the preservation of neurofilament H in the CNS of EAE animals in an embodiment of the invention.

SJL animals are induced for disease by the passive transfer of encephalitogenic T cells as described in Example 1. Animals are placed on either Tempol or control feed. The spinal cords of the animals are analyzed 90 days post transfer.

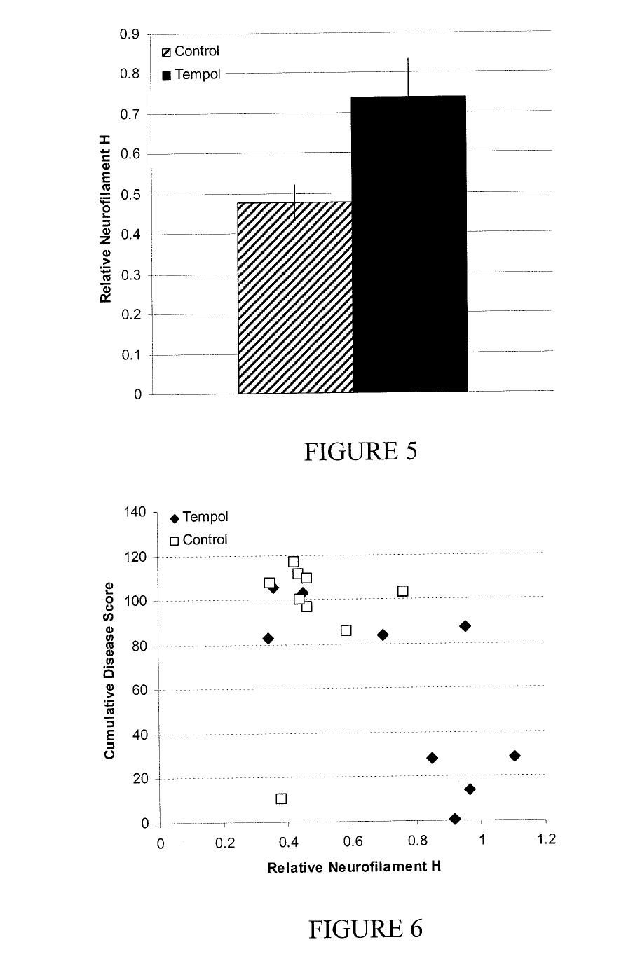

Neurofilament staining is representative of the presence of axons. During autoimmune or demyelinating processes, the loss of neurofilament H has been associated with axonal loss. Animals on Tempol feed show a greater preservation of neurofilament H suggesting lesser axonal loss than animals on control feed (FIG. 5 and Table 3).

TABLE 3
Tempol

| | | | | | | | | | |
|---|---|---|---|---|---|---|---|---|---|
| average | 0.96519 | 0.44868 | 0.70000 | 0.35894 | 0.84991 | 0.34151 | 0.91708 | 0.95532 | 1.10406 |
| cumulative | 14 | 103.25 | 83.75 | 105.25 | 28.25 | 83 | 0.5 | 87.25 | 28.75 |
| disease score | | | | | | | | | |
| peak disease | 2.25 | 3.5 | 2.75 | 3.5 | 2.5 | 3.5 | 0 | 3.5 | 2.25 |
| score | | | | | | | | | |

Control

| | | | | | | | | | |
|---|---|---|---|---|---|---|---|---|---|
| average | 0.46499 | 0.76515 | 0.58474 | 0.43556 | 0.42431 | 0.46334 | 0.44067 | 0.34896 | 0.37887 |
| cumulative | 96.75 | 103.25 | 86 | 111.5 | 117 | 109.25 | 100 | 107.5 | 10.25 |
| disease score | | | | | | | | | |
| peak disease | 3.25 | 3.75 | 2.75 | 3.5 | 4 | 3.25 | 2.75 | 3.25 | 2 |
| score | | | | | | | | | |

The higher amounts of neurofilament H in Tempol-fed animals correlate with the reduced disease severity/cumulative disease score over the 60 days animals are followed (FIG. 6).

### Example 4

This example demonstrates that a nitroxide compound of formula I reduces the degree of edema associated with inflammation and tissue damage in an animal in an embodiment of the invention.

SJL animals are induced for disease by the passive transfer of encephalitogenic T cells as described in Example 1. Animals are placed on either Tempol or control feed. In a spinal cord histogram, T₂ relaxation times typically increase due to inflammation and edema during CNS autoimmunity/disease during EAE. In this experiment, the histogram shows that Tempol-fed animals have lower T₂ relaxation times, thereby indicating less edema, than control-fed animals. In the control-fed animals, the spinal cord T₂ relaxation times shifted to higher values due to edema and inflammation.

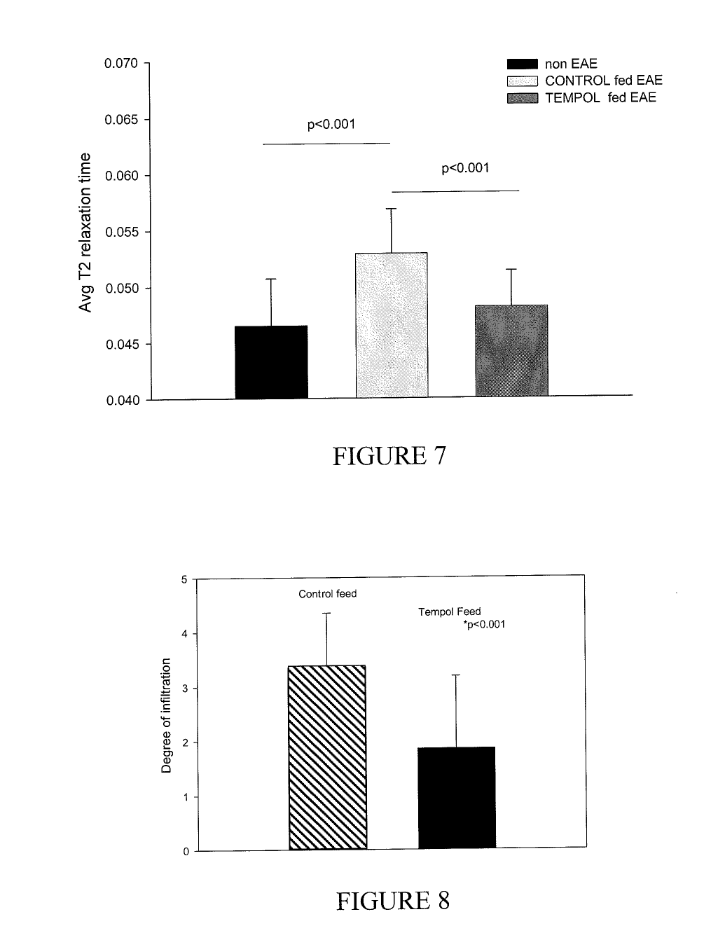

The average T2 relaxation times are measured for SJL passively-induced EAE animals on Tempol 2 weeks prior to T cell transfer. Five scans are collected per animal, which are pooled from: 2 control animals (10 total), 4 control-fed EAE animals (20 total), and 4 Tempol-fed EAE animals (20 total). FIG. 7 shows that EAE animals exhibit significantly increased T2 relaxation times. Thus, Tempol limits (e.g., reduces) the edema and inflammation in animals undergoing passive EAE to levels observed in healthy animals.

Animals induced passively for EAE with PLP-specific T cells are examined at peak disease for assessment of inflammatory infiltrates. The spinal cords from 5 animals per group (control-fed and Tempol-fed) are examined at 11 levels spanning the entire cord and assessed according to the following scale and an average inflammatory score is calculated per animal. Degree of inflammation: 1=1-2 mild infiltrates, little to no infiltration; 2=3+ infiltrates, +/− infiltration; 3=1-2 moderate infiltrates, 1-2 mild little/mild infiltrates; 4=3+ moderate infiltrates, or 1 severe/broad with or without mild infiltrates, extensive infiltration to parenchyma present; 5=2+ severe/broad parenchyma infiltrative, with or without additional mild/moderate associated infiltrates to parenchyma.

The degree of inflammation is averaged for the study groups and compared as shown in FIG. 8. As seen in FIG. 8, Tempol limits the inflammatory infiltrates observed at peak disease in animals passively induced for EAE.

### Example 5

This example demonstrates that a nitroxide compound of formula I reduces the degree of leukocytic infiltration in an animal in an embodiment of the invention.

C57BL/6J animals are actively induced for disease as described in Example 1. Animals are placed on either Tempol or control feed, and the number of leukocytes are counted via flow cytometry.

TABLE 4

| | Cell # | % | % total | | | | CD4:CD8 |
|---|---|---|---|---|---|---|---|
| Treatment | (×10⁶) | microglia | CD45 hi | % mac/gran | % B cell | % CD3 | ratio |
| control | 1.8 | 38.3 | 51.8 | 13.0 | 4.0 | 36.0 | 4.0 |
| control | 1.4 | 18.5 | 66.7 | 28.3 | 8.3 | 31.2 | 5.6 |
| Tempol | 1.0 | 68.5 | 21.0 | 10.1 | 1.3 | 11.1 | 1.7 |
| Tempol | 1.0 | 55.6 | 33.8 | 20.2 | 2.8 | 11.2 | 1.5 |

As seen in Table 4, Tempol limits the inflammatory infiltrates observed at peak disease in animals actively induced for EAE.

It also is observed that Tempol reduces the degree of microglial activation (indicated by upregulation of MHC class II expression) observed in EAE compared to animals on control feed. These results indicate that a compound of formula I reduces immune activation within the CNS.

### Example 6

This example demonstrates that a nitroxide compound of formula I does not reduce the generation of myelin-specific T cells in an animal.

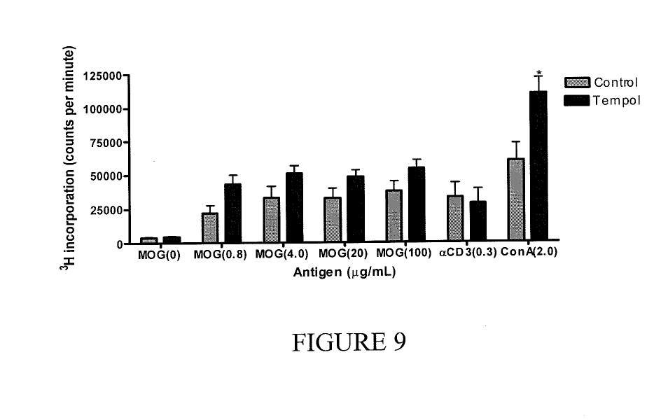

Animals on control or Tempol feed are immunized with a myelin antigen, and the spleens are tested 20 days later for recall to antigen. Three animals per group are tested and the results are pooled (FIG. 9). As seen in FIG. 9, Tempol does not reduce the generation of myelin-specific T cells as assessed by proliferative responses.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a disease involving myelin and/or axonal loss in a mammal comprising administering to the mammal an effective amount of a compound of formula I 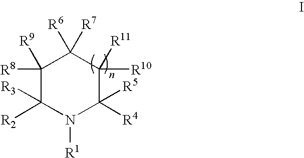
wherein
R¹ is selected from the group consisting of OH, OZ, O, and ═O,
wherein Z is selected from the group consisting of C₁₋₁₂ alkyl, C₂₋₁₂ alkenyl, C₃₋₈ cycloalkyl, C₃₋₈ heterocycloalkyl, and C₆₋₃₀ aryl;
R², R³, R⁴, and R⁵ are the same or different and are selected from the group consisting of hydrogen, C₁₋₁₂ alkyl, C₂₋₁₂ alkenyl, and C₂₋₁₂ alkynyl;
R⁶ and R⁷ are the same or different and are selected from the group consisting of hydrogen, halogen, hydroxyl, thiol, cyano, —NCS, C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino, or
R⁶ and R⁷ together form ═O;
R⁸, R⁹, R¹⁰, and R¹¹ are the same or different and are selected from the group consisting of hydrogen, halogen, hydroxyl, thiol, cyano, —NCS, C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino;
optionally one of R⁶ and R⁷ and one of R⁸ and R⁹ can be absent such that a double bond joins the two carbon atoms to which the remaining of one of R⁶ and R⁷ and one of R⁸ and R⁹ are attached; and
n is 0 or 1;
provided that the disease is not ataxia telangiectasia (AT).

2. The method of claim 1, wherein the disease is a demyelinating disease.

3. The method of claim 1, wherein the disease involves axonal damage or impairment.

4. The method of claim 1, wherein the disease is an inflammatory disease.

5. The method of claim 1, wherein an autoimmune component of the disease is treated.

6. The method of claim 1, wherein the disease is multiple sclerosis (MS), optic neuritis, Devic's disease (neuromyelitis optica), transverse myelitis, acute MS (Marburg variant), Balo's concentric sclerosis, Guillain-Barré syndrome, acute disseminated encephalomyelitis (ADEM), adrenoleukodystrophy, or adrenomyeloneuropathy.

7. The method of claim 6, wherein the disease is multiple sclerosis (MS).

8. A method of treating neurodegeneration associated with inflammation in a mammal comprising administering to the mammal an effective amount of a compound of formula I 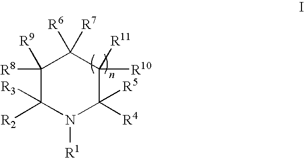
wherein
R¹ is selected from the group consisting of OH, OZ, O, and ═O,
wherein Z is selected from the group consisting of C₁₋₁₂ alkyl, C₂₋₁₂ alkenyl, C₃₋₈ cycloalkyl, C₃₋₈ heterocycloalkyl, and C₆₋₃₀ aryl;
R², R³, R⁴, and R⁵ are the same or different and are selected from the group consisting of hydrogen, C₁₋₁₂ alkyl, C₂₋₁₂ alkenyl, and C₂₋₁₂ alkynyl;
R⁶ and R⁷ are the same or different and are selected from the group consisting of hydrogen, halogen, hydroxyl, thiol, cyano, —NCS, C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino, or
R⁶ and R⁷ together form ═O;
R⁸, R⁹, R¹⁰, and R¹¹ are the same or different and are selected from the group consisting of hydrogen, halogen, hydroxyl, thiol, cyano, —NCS, C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino;
optionally one of R⁶ and R⁷ and one of R⁸ and R⁹ can be absent such that a double bond joins the two carbon atoms to which the remaining of one of R⁶ and R⁷ and one of R⁸ and R⁹ are attached; and
n is 0 or 1;
provided that the neurodegeneration is not caused by ataxia telangiectasia (AT).

9. The method of claim 8, wherein the compound of formula I provides protection against onset and progression of neurodegeneration of the central nervous system (CNS).

10. The method of claim 8, wherein the neurodegeneration is caused by multiple sclerosis (MS), optic neuritis, Devic's disease (neuromyelitis optica), transverse myelitis, acute MS (Marburg variant), Balo's concentric sclerosis, Guillain-Barré syndrome, acute disseminated encephalomyelitis (ADEM), adrenoleukodystrophy, or adrenomyeloneuropathy.

11. The method of claim 10, wherein the neurodegeneration is caused by multiple sclerosis (MS).

12. A method of reducing myelin and/or axonal loss in a mammal comprising administering to the mammal an effective amount of a compound of formula I 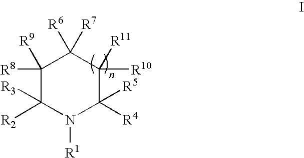
wherein
R¹ is selected from the group consisting of OH, OZ, O, and ═O,
wherein Z is selected from the group consisting of C₁₋₁₂ alkyl, C₂₋₁₂ alkenyl, C₃₋₈ cycloalkyl, C₃₋₈ heterocycloalkyl, and C₆₋₃₀ aryl;
R², R³, R⁴, and R⁵ are the same or different and are selected from the group consisting of hydrogen, C₁₋₁₂ alkyl, C₂₋₁₂ alkenyl, and C₂₋₁₂ alkynyl;
hydrogen, halogen, hydroxyl, thiol, cyano, —NCS, C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino, or
R⁶ and R⁷ together form ═O;
R⁸, R⁹, R¹⁰, and R¹¹ are the same or different and are selected from the group consisting of hydrogen, halogen, hydroxyl, thiol, cyano, —NCS, C₁₋₁₂ alkyl, C₂₋₁₂ alkenyl, C₃₋₈ cycloalkyl, C₃₋₈ heterocycloalkyl, C₆₋₃₀ aryl, C₁₋₁₂ alkoxy, C₁₋₁₂ alkylthio, amino, alkylamino, dialkylamino, arylamino, diarylamino, alkylsulfonyloxy, carboxyl, alkylcarbonyl, arylcarbonyl, hydroxyalkyl, mercaptoalkyl, carboxyalkyl, carboxyaryl, alkylcarbonylalkyl, alkylcarbonylaryl, aminoalkyl, alkylaminoalkyl, dialkylaminoalkyl, arylaminoalkyl, diarylaminoalkyl, alkylcarbonylamino, and haloalkylcarbonylamino;
optionally one of R⁶ and R⁷ and one of R⁸ and R⁹ can be absent such that a double bond joins the two carbon atoms to which the remaining of one of R⁶ and R⁷ and one of R⁸ and R⁹ are attached; and
n is 0 or 1.

13. The method of claim 1, wherein R¹ is O.

14. The method of claim 1, wherein R², R³, R⁴, and R⁵ are C₁₋₁₂ alkyl.

15. The method of claim 1, wherein R², R³, R⁴, and R⁵ are C₁₋₄ alkyl.

16. The method of claim 1, wherein R², R³, R⁴, and R⁵ are methyl.

17. The method of claim 1, wherein R⁷ is selected from the group consisting of hydrogen, halogen, hydroxyl, and C₁₋₁₂ alkyl.

18. The method of claim 1, wherein R⁷ is hydrogen.

19. The method of claim 1, wherein R⁸, R⁹, R¹⁰, and R¹¹ are the same or different and each is selected from the group consisting of hydrogen, halogen, hydroxyl, and C₁₋₁₂ alkyl.

20. The method of claim 1, wherein R⁸, R⁹, R¹⁰, and R¹¹ are hydrogen.

21. The method of claim 1, wherein R⁶ is hydrogen, hydroxyl, C₁₋₁₂ alkyl, C₁₋₁₂ alkoxy, cyano, isothiocyanato, amino, carboxy, alkylcarbonylamino, haloalkylcarbonylamino, or alkylsulfonyloxy.

22. The method of claim 1, wherein R⁶ is hydroxyl.

23. The method of claim 1, wherein n is 1.

24. The method of claim 1, wherein the compound of formula I is selected from the group consisting of
4-hydroxy-2,2,6,6-tetramethylpiperidine-1-oxyl (Tempol),
2,2,6,6-tetramethylpiperidine-1-oxyl (Tempo),
4-methoxy-2,2,6,6-tetramethyl-1-piperidine-1-oxyl,
4-carboxy-2,2,6,6-tetramethyl-1-piperidine-1-oxyl,
4-oxo-2,2,6,6-tetramethyl-1-piperidine-1-oxyl,
4-amino-2,2,6,6-tetramethyl-1-piperidine-1-oxyl,
4-cyano-2,2,6,6-tetramethyl-1-piperidine-1-oxyl,
4-isocyanato-2,2,6,6-tetramethyl-1-piperidine-1-oxyl,
4-acetamido-2,2,6,6-tetramethyl-1-piperidine-1-oxyl,
4-(2-bromoacetamido)-2,2,6,6-tetramethyl-1-piperidine-1-oxyl,
4-(2-chloroacetamido)-2,2,6,6-tetramethyl-1-piperidine-1-oxyl,
4-(2-iodoacetamido)-2,2,6,6-tetramethyl-1-piperidine-1-oxyl, and
4-(methylsulfonyloxy)-2,2,6,6-tetramethyl-1-piperidine-1-oxyl.

25. The method of claim 1, wherein the compound of formula I is 4-hydroxy-2,2,6,6-tetramethylpiperidine-1-oxyl (Tempol).

26. The method of claim 1, wherein the mammal is a human.

27. The method of claim 1, wherein the compound of formula I is administered orally.

28. The method of claim 27, wherein the compound of formula I is administered with food.
